(19) United States
(12) Patent Application Publication
(10) Pub. No.: US 2014/0263695 A1
(43) Pub. Date: Sep. 18, 2014

(54) METHOD AND APPARATUS FOR ATOMIZING AND VAPORIZING LIQUID

(71) Applicant: KING ABDULLAH UNIVERSITY OF SCIENCE AND TECHNOLOGY, Thuwal (SA)

(72) Inventors: Amit Lal, Ithaka, NY (US); Abdulilah M. Mayet, Thuwal (SA)

(73) Assignee: KING ABDULLAH UNIVERSITY OF SCIENCE AND TECHNOLOGY, Thuwal (SA)

(21) Appl. No.: 13/800,644

(22) Filed: Mar. 13, 2013

(51) Int. Cl. A61L 9/03

(52) U.S. Cl. CPC A61L 9/037
USPC 239/4; 239/136; 239/102.2

(57) ABSTRACT

A method and apparatus for atomizing and vaporizing liquid is described. An apparatus having an ejector configured to eject one or more droplets of liquid may be inserted into a reservoir containing liquid. The ejector may have a vibrating device that vibrates the ejector and causes liquid to move from the reservoir up through the ejector and out through an orifice located on the top of the ejector. The one or more droplets of liquid ejected from the ejector may be heated and vaporized into the air.

### FIELD OF THE INVENTION

The exemplary embodiments of the invention relate generally to an apparatus and method for atomizing and vaporizing liquid.

### BACKGROUND OF THE INVENTION

Commercial air fresheners generally fall into two categories, mechanical atomizers, which use a mechanical motor operated by a power source to push a valve and allow compressed perfume inside a can to be released, and perfume evaporator and burners, which use an open flame to evaporate perfume carried by an additive such as wax or other combustible material.

Commercial air fresheners have drawbacks. For example, commercial air fresheners may waste energy and may cause pollution. Mechanical atomizers can consume a lot of power while operating the motor and spray the perfume in form of droplets that do not dissolve into the air due to the high surface tension and low vapor pressure of the fluid. Perfume evaporator and burners may cause air pollution due to the flame and carbon dioxide generation. Additionally, perfume evaporators and burners may change the chemistry of the perfume due to the high temperature, which may be harmful if inhaled.

Another drawback of commercial air fresheners is the short duration of the air freshening effect of the products. Commercial air fresheners generally deliver large droplets of fluid into the air, which can fall to the ground due to gravity, and settle to the ground, and result in slow perfume dispersal requiring even more perfume to be delivered. Additionally, the current commercial electrical air fresheners are often bulky and inefficient devices, which consume a large amount of energy and waste fragrance by dispensing large droplets of fragrance into the air. Thus, there is a need for an improved air freshening device.

### SUMMARY

In general, an apparatus for vaporizing and atomizing liquid is described. The apparatus may include an ejector having an orifice at a top portion of the ejector. The ejector may be configured to eject one or more droplets of liquid from the orifice. The apparatus may include a capillary tube surrounding at least a portion of the ejector. The capillary tube may be configured to extend into a container having liquid. An outer sheath may be configured to surround at least a portion of the ejector and at least a portion of the capillary tube. At least a portion of the outer sheath may extend above the ejector orifice. A heater may be mounted to an inner surface of the outer sheath and positioned above the ejector orifice to vaporize droplets of liquid ejected from the orifice of the ejector. The apparatus may include a vibrating device coupled to the ejector, which is configured to provide longitudinal vibration to the ejector. The apparatus may include a power source to provide power to the vibrating device. In at least one embodiment, the apparatus is configured to be inserted into a decorative object.

In certain embodiments, a method for vaporizing and atomizing liquid is described. An ejecting apparatus may be inserted into a reservoir containing liquid. The ejecting apparatus may be longitudinally vibrated, which causes fluid to be displaced longitudinally through a bottom orifice of the ejector and expelled from a top orifice of the ejector. The fluid ejected from the ejecting apparatus may be heated to vaporize the ejected fluid into the air.

One object of the invention is to dissolve emitted fragrance in the air, which will prolong the air freshening effect. Another object of the invention is to create a product that is more sustainable and more cost effective than current commercial air fresheners. A further object of the invention is to release fragrance into the air without changing the chemical composition of the fragrance.

Other aspects, embodiments, and features will be apparent from the following description, the drawings, and the claims.

### BRIEF DESCRIPTION OF THE DRAWINGS

FIG. 1 illustrates a fluid atomizer in accordance with at least one embodiment of the invention.

FIG. 2 illustrates an ejector in accordance with at least one embodiment of the invention.

FIG. 3 illustrates a portion of the fluid atomizer in accordance with at least one embodiment of the invention.

FIG. 4 illustrates a top view of a filter in accordance with at least one embodiment of the invention.

FIG. 5 illustrates a method of atomizing fluid in accordance with at least one embodiment of the invention.

Before the embodiments of the invention are explained in detail, it is to be understood that the invention is not limited in its application to the details of construction and the arrangement of the components set forth in the following description or illustrated in the drawings. The invention is capable of other embodiments and of being practiced or being carried out in various ways.

### DETAILED DESCRIPTION

An apparatus and a method for atomizing and vaporizing liquid is described herein. An apparatus including an ejector configured to eject one or more droplets of liquid from an orifice at the top of the ejector is inserted into a liquid reservoir. Vibration may be applied to the ejector, which causes liquid to pump into the ejector and out through the orifice at the top of the ejector, as described in U.S. Pat. No. 6,923,790 entitled “Ultrasonically Actuated Needle Pump,” which is incorporated by reference herein. A heater positioned above the orifice of the ejector may heat the ejected droplets to atomize and vaporize the liquid faster than at ambient temperature. The heating can occur by the droplets impacting the hot surface, or experiencing the higher temperature in the air as the droplet goes through the hot air.

Referring to the Figures wherein like numerals indicate like elements, FIG. 1 depicts an apparatus 100 for atomizing and vaporizing liquid. In at least one embodiment, the apparatus 100 is an air freshener. As shown in FIG. 2, the apparatus 100 may include an ejector 101, for ejecting one or more droplets of liquid. The ejector 101 may eject a single droplet at one time or a plurality of droplets. Alternatively or additionally, the ejector 101 may eject a stream of droplets in series.

The ejector 101 may be any suitable size or shape to eject one or more droplets of liquid. In at least one embodiment, the ejector has a diameter of 0.2 to 3 mm, for example 1 mm. The ejector 101 may include an orifice 102 at the top portion of the ejector 101. The orifice 102 may be any suitable size for ejecting small droplets of liquid or fragrance from the ejector 101. The inner diameter of orifice 102 may range from 0.1-300 μm and have an outer diameter in the range of 0.2 to 400 μm. In at least one embodiment, the diameter of the orifice 102 is 10 μm. In at least one embodiment, the ejector 101 is configured to emit sub-micron size droplets of liquid. The ejector 101 may also include a tapered portion 103 adjacent the orifice 102. The ejector may be made of any suitable material, such as glass. The ejector 101 may also have a bottom orifice at a distal end of the ejector 101. The bottom orifice may be any suitable diameter. In at least one embodiment, the diameter of the bottom orifice is the same diameter as the diameter of the ejector 101 (i.e. 1 mm).

The ejector 101 may be coupled or attached to a vibrating device 115. The vibrating device 115 may be attached to the ejector in any suitable manner, such as by an adhesive. The vibrating device 115 may be any type of vibrating device, including a piezoelectric plate made from a lead zirconate titanate material. The vibrating device 115 may be any suitable size or shape. For example, the vibrating device 115 may be circular, rectangular, or square. The vibrating device 115 may have a length corresponding to the length of the ejector 101 or have a length that is smaller than the length of the ejector 101. In at least one embodiment, the width of the vibrating device 115 is between 2 and 3 mm. In at least one embodiment, the width of the vibrating device 115 may be 2.5 mm. In another embodiment, the length of the vibrating device 115 is between 5 and 20 mm. In at least one embodiment, the length of the vibrating device 115 is 10 mm. The vibrating device 115 may provide an ultrasonic vibration at any suitable range, for example, 150-170 kHz. In at least one embodiment, the vibration is approximately 160 kHz.

Vibration may be applied to the ejector 101 in one or both of a horizontal direction or a vertical direction, or in a twisting motion, or a combination of these motions. The vibrating device 115 may include electrodes 116 that are attached to a microcontroller 112, as illustrated in FIG. 1. The microcontroller 112 may control the power supplied to the vibrating device 115. The microcontroller 112 may include a power source, which provides power to the microcontroller 112. In at least one embodiment, the power source is a battery. In alternative embodiments, the power source may be a fuel cell, a solar cell, or any other suitable electrical power device. The microcontroller 112 may also include a solar cell to recharge the power source. The solar cell may be incorporated into the microcontroller 112 or attached to the microcontroller 112. Alternatively, the solar cell may be physically separate from the microcontroller 112 but electrically coupled to the microcontroller 112. The microcontroller 112 may also include a timing device to control the time and duration that the ejector 101 ejects liquid.

As illustrated in FIG. 3, the apparatus 100 may include a capillary 104. The capillary 104 may be made of any suitable material for directing liquids into the ejector 101. For example, the capillary 104 may be made of glass or plastic. The capillary 104 may be attached to at least a portion of the ejector 101. For example, the capillary 104 may be attached to a bottom portion of the ejector 101. The capillary 104 may extend beyond a bottom end of the ejector 101. The capillary 104 may be any suitable size and shape. For example, the capillary 104 may be a circular capillary that extends around the outside diameter of the ejector 101. Alternatively, the capillary 104 may be configured to fit within an interior portion of the ejector 101. In at least one embodiment, the capillary 104 includes one or more orifices 110 on a sidewall of the capillary 104. The orifices 110 may maintain the level of liquid in the capillary 104. The capillary 104 may be attached to the ejector 101 in any suitable manner. For example, the capillary 104 may be attached to the ejector 101 by a semi porous clamp holder 107. The semiporous clamp can be shaped out of materials such as a sponge, or a porous Teflon, or made of a solid with machined micro holes using a laser or water drilling. The semisporosity provides a low-acoustic impedance way to provide mechanical support to the vibrating structure so as to minimize the coupling to the external tube holder.

The ejector 101 may include a filter 108 attached to a bottom end of the ejector 101. The bottom end of the ejector 101 may be open to allow liquid to flow upward into the ejector 101 and out through the orifice 102. The filter 108, as illustrated in FIG. 4, may include a porous membrane 109 to filter the liquid. The porous membrane holes will be in the size of 0.1 to 10 microns such that any large particles in the fluid cannot enter the capillary leading to clogging the capillary. The porous membrane 109 may include a plurality of small holes to allow liquid to flow through the filter 108. The filter 108 may be any suitable shape or size and may be configured to surround at least a portion of the ejector 101. The porous membrane 109 may include any size and any number of holes to allow liquid to flow through the filter 108.

Referring back to FIG. 3, the apparatus 100 may include an outer sheath 111 that extends above the orifice 102 of the ejector 101. The outer sheath 111 may be any suitable shape and size. In at least one embodiment, the outer sheath 111 may be a tubular structure surrounding the ejector 101. The outer sheath 111 may be attached to an outer surface of the capillary 104 or may be attached to the ejector 101. The outer sheath 111 may include one or more resistive heaters 106 on an inner surface of the outer sheath 111. The resistive heater 106 may be positioned near and adjacent the orifice 102 of the ejector 101. The resistive heaters 106 may be powered by the microcontroller 112. The resistive heaters 106 may heat droplets of liquid or aqueous fragrance emitted by the ejector 101, which causes the droplets of liquid to vaporize or atomize and be dispersed into the air. In at least one embodiment, the resistive heaters 106 do not alter the chemical make-up of the liquid or fragrance being emitted by the ejector 101.

The apparatus 100 may include a coupling device 105. The coupling device 105 may be any suitable material for coupling the apparatus 100 to a container 114. For example, the coupling device 105 may be made of cork or rubber. The coupling device 105 may be attached to the outer sheath 111 and configured to surround at least a portion of the outer sheath 111. In at least one embodiment, the coupling device 105 is configured to attach the apparatus 100 to an opening of a container 114. The coupling device 105 may be any suitable shape or size to attach the apparatus 100 to the container 114. For example, the coupling device may be in the shape of a wedge, a circle, etc. or may be a plurality of shapes.

Referring back to FIG. 1, the apparatus 100 may include a fluidic capsule 113. The fluidic capsule 113 may be attached to the capillary 104 and/or the outer sheath 111. The fluidic capsule 113 may be configured to extend downwardly from the outer sheath 111 into a container 114 of liquid or fragrance. The fluidic capsule 113 may be configured to draw liquid up from the bottom of a container toward the capillary 104 and ejector 101. The vibrating device 115 may vibrate the ejector 101 and the capillary 104, which creates suction and acts as a pump to pump liquid or fragrance up through the fluidic capsule 113 through the filter 108, up through the ejector 101 and out through the orifice 102.

Alternatively, the capillary 104 may extend into the container 114 of liquid or fragrance and be configured to draw liquid into the ejector 101 from the container 114. In this embodiment, the vibrating device 115 may vibrate the ejector 101 and the capillary 104, which creates suction and acts as a pump to pump the liquid or fragrance up through the capillary 104, the filter 108, the ejector 101, and out through the orifice 102.

The vibrating device 115 may operate at any suitable frequency range to draw liquid up through the capillary 104 and ejector 102 of the apparatus 100. In at least one embodiment, the vibrating device operates at approximately 160 kHz, which is the natural frequency of a glass ejector 101.

In at least one embodiment, the apparatus 100 is configured to fit into a decorative object. For example, the apparatus 100 may fit into a flower shaped object and emit a fragrance. In this embodiment, the fragrance emitted may replicate the smell of fresh flowers. Alternatively, the apparatus 100 may fit into other decorative objects such as candle holders, vases, and the like.

FIG. 5 depicts a method of atomizing liquid or fragrance. The method of atomizing liquid or fragrance described in FIG. 5 may utilize one or more aspects of the apparatus 100 described above. As shown in Step 501, an ejecting apparatus, which may include the features of apparatus 100 described above, is inserted into a reservoir containing liquid. In Step 502, an ejector within the ejecting apparatus is vibrated. The ejector may be vibrated in either the horizontal or longitudinal direction or twisting direction, or some combination of the three directions of motion. Once vibration is applied to the ejector, fluid may be displaced longitudinally through a bottom orifice of the ejector and expelled from a top orifice of the ejector. In Step 503, the fluid ejected from the ejecting apparatus may be heated to vaporize the ejected fluid into the air.

Variations and modifications of the foregoing are within the scope of the present invention. For example, one of skill in the art will understand that multiples of the described components may be used in stores and in various configurations. The present invention is therefore not to be limited to a single system, nor the upright pusher configuration, depicted in the Figures, as the system is simply illustrative of the features, teachings and principles of the invention. It should further be understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

What is claimed is:

1. An apparatus comprising:
an ejector having an orifice at a top portion, the ejector is configured to eject droplets of liquid from the orifice;
a capillary tube surrounding at least a portion of the ejector, the capillary tube configured to extend into a container;
an outer sheath configured to surround at least a portion of the ejector and at least a portion of the capillary tube, wherein at least a portion of the outer sheath extends above the ejector orifice; and
a heater mounted to an inner surface of the outer sheath,
wherein the heater is positioned above the ejector orifice.

2. The apparatus of claim 1, further comprising:
a filter attached to a lower portion of the ejector, the lower portion opposition the top portion.

3. The apparatus of claim 1, further comprising:
a vibrating device coupled to the ejector.

4. The apparatus of claim 1, further comprising:
a coupling device attached to a lower portion of the outer sheath, the coupling device configured to attach the apparatus to a container.

5. The apparatus of claim 3, wherein the vibrating device includes a piezoelectric plate.

6. The apparatus of claim 4, wherein the coupling device includes one or more pieces of cork.

7. The apparatus of claim 3, further comprising:
a microcontroller having a power source, the microcontroller configured to provide power to the vibrating device.

8. The apparatus of claim 7, further comprising:
a timing device in the microcontroller, the timing device configured to control the time and duration of liquid ejected from the ejector.

9. The apparatus of claim 7, further comprising:
a solar cell configured to charge the power source in the microcontroller.

10. The apparatus of claim 1, further comprising a fluidic capsule attached to the capillary tube.

11. An apparatus comprising:
an ejector having an orifice at a top portion, the ejector is configured to eject droplets of liquid from the orifice;
a filter attached to a lower portion of the ejector;
a capillary tube surrounding at least a portion of the ejector, the capillary tube configured to extend into a container;
an outer sheath configured to surround at least a portion of the ejector and at least a portion of the capillary tube, wherein at least a portion of the outer sheath extends above the ejector orifice;
a heater mounted to an inner surface of the outer sheath; and
a fluidic capsule attached to the capillary tube,
wherein the heater is positioned above the ejector orifice.

12. The apparatus of claim 11, further comprising:
a vibrating device coupled to the ejector.

13. The apparatus of claim 11, further comprising:
a coupling device attached to a lower portion of the outer sheath, the coupling device configured to attach the apparatus to a container.

14. The apparatus of claim 12, further comprising:
a microcontroller having a power source, the microcontroller configured to provide power to the vibrating device.

15. The apparatus of claim 14, further comprising:
a timing device in the microcontroller, the timing device configured to control the time and duration of liquid ejected from the ejector.

16. The apparatus of claim 11, further comprising:
a coupling device attached to a lower portion of the outer sheath, the coupling device configured to attach the apparatus to a container.

17. A method comprising:
inserting an ejector apparatus into a container filled with liquid;
applying longitudinal vibration to an ejector in the ejector apparatus, wherein applying longitudinal vibration to the ejector causes liquid to enter a bottom orifice of the ejector and rise up longitudinally through the ejector and eject from a top orifice of the ejector; and
heating the ejected liquid to vaporize the ejected liquid into the air, wherein the ejected liquid is heated by a heater mounted on an inner surface of an outer sheath that is coupled to the ejector.

18. The method of claim 17, wherein the longitudinal vibration is applied at approximately 150-170 kHz.

19. The method of claim 17, wherein an inner diameter of the top orifice ranges from 0.1-30 microns.

20. The method of claim 17, wherein a diameter of the bottom orifice is approximately 1 mm.
